(19) United States
(12) Patent Application Publication
(10) Pub. No.: US 2008/0299221 A1
(43) Pub. Date: Dec. 4, 2008

(54) Method for Decreasing Postprandial Glucose Excursion

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 12/096,529

(22) PCT Filed: Dec. 12, 2005
(86) PCT No.: PCT/RU2005/000640

§ 371 Date: Aug. 13, 2008

(51) Int. Cl. A61K 33/00; A61P 7/00

(52) U.S. Cl. 424/600

(57) ABSTRACT

The invention relates to the method for decreasing postprandial glucose excursion in a mammal in need thereof comprising a step of administering to said mammal an effective amount of water comprising from 99.76 to 99.99 molecular % of isotopologue ¹H₂0 at 0 to 240 min from the start of ingestion of a meal. Preferably, the effective amount water comprising from 99.76 to 99.99 molecular % of isotopologue ¹H₂¹⁶0 is administered orally. Preferably, the effective amount of the water comprising from about 99.76 to about 99.99 molecular % of isotopologue ¹H₂¹⁶0 is 0.1 to 20 g/kg body weight of a mammal. Further, invention relates to the medical food for decreasing postprandial glucose excursion in a mammal in need thereof which comprises water comprising from about 99.76 to about 99.99 molecular % of isotopologue ¹H₂¹⁶0. Further, invention relates to use of water comprising from about 99.76 to about 99.99 molecular % of isotopologue ¹H₂¹⁶0 for the manufacture of a medical food for decreasing postprandial glucose excursion in a mammal in need thereof. Preferably, the mammal is human.

### TECHNICAL FIELD

The present invention is in the field of healthcare. More specifically, the invention relates to method for decreasing postprandial glucose excursion in mammals, preferably in a human.

### BACKGROUND ART

Epidemiological studies have underlined the role of postprandial (after a meal) blood glucose (PPG) as a significant risk factor for cardiovascular diseases (CVD) and type 2 diabetes.

PPG is a risk factor of CVD among apparently healthy individuals. Levitan E B, et al., Arch Intern Med. (2004) 164:2147-2155. PPG is an independent risk factor for increased carotid intima-media thickness in non-diabetic individuals. Hanefeld M et al. Atherosclerosis. (1999) 144(1):229-35. PPG is a risk factor for mortality in middle-aged nondiabetic men. Balkau et al., Diabetes Care 1998, 21(3):360-7. There is a direct link between PPG and endothelial dysfunction. Ceriello A et al., Circulation 2002; 106:1211-1218. Ceriello A et al., Diabet Med. 2004, 21(2):171-5. Two-hour PPG >7.8 mmol/L significantly increases risk of cardiovascular disease mortality in individuals with normal fasting glucose (<6.1 mmol/L) and normal HbA_{1C} (<6.1%) levels. Gerich J E, Arch Intern Med. 2003 163(11):1306-1316. Postprandial hyperglycemia has a direct toxic effect on arteries. Ceriello A et al., Diabetic Medicine 2004; 21:171-175. Arterial stiffness, a known marker of atherosclerosis, significantly correlated with two-hour PPG. Oshita K et al., Diabet Med 2004; 21:636-639. Targeting PPG can lower HbA_{1c} effectively. Christiansen J S et al., Diabetes, Obesity and Metabolism 2003; 5: 446-454. Therapy targeted at PPG improves atherosclerotic markers and reduces cardiovascular events. Chaisson J et al., Lancet 2002; 359:2072-2077. Chaisson J et al., JAMA 2003; 290:486-494. Controlling PPG can prevent progression of atherosclerosis. Esposito K et al., Circulation 2004; 110(2): 214-219. Targeting PPG slowed the progression of atherosclerosis in carotid arteries. Hanefeld M et al., Stroke 2004; 35:1073-1078. Targeting PPG lowered a relative risk of myocardial infarction of 64% and any cardiovascular events of 35%. Hanefeld M et al., Eur Heart J 2004; 25(1):10-16. PPG is a more significant contributor to HbA_{1c} than fasting glucose. Avignon A et al., Diabetes Care. 1997, 20(12):1822-6. Bastyr E et al., Diabetes Care 2000, 23:1236-41.

Today, the postprandial phase lasts about 20 hours per day in western societies with a dinner culture. Thus, there is a great need for a safe, effective agent for decreasing postprandial glucose excursion in apparently healthy individuals, overweight persons, obese persons or persons with impaired glucose tolerance to prevent risk of cardiovascular disease and type 2 diabetes. Also, there is a great need for a safe, effective agent for reducing postprandial hyperglycemia in diabetic subjects to reduce risk of cardiovascular disease.

Decreasing postprandial glucose excursion c an be achieved with low carbohydrate diet that restricts carbohydrate intake. Another way to decrease postprandial glucose excursions in apparently healthy individuals is the use of food with low glycemic index. Brand-Miller et al., Am. J. Clin. Nutr. 76(1), 281S-285S, 2002. Hodge A M et al., Diabetes Care 27:2701-2706, 2004. Ludwig D S, JAMA. 2002, 287(18): 2414-23. However, individuals frequently do not follow these diets since the dietetic restrictions are uncomfortable in daily practice.

Reducing postprandial hyperglycemia in persons with impaired glucose tolerance and type 2 diabetes can be achieved with insulin therapy. Christiansen J S et al., Diabetes Obes Metab. 2003, 5(6):446-54. Hanefeld M et al., Eur Heart J. 2004, 25(1): 10-6. However, such therapy does not acceptable for apparently healthy individuals.

It is known that natural water is a composition of nine water isotopologues (¹H₂¹⁶O, ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O) formed by stable isotopes of hydrogen (¹H and ²H) and oxygen (¹⁶O, ¹⁷O, ¹⁸O), wherein the level of light water isotopologue ¹H₂¹⁶O is about 99.7317 molecular % (Vienna Standard Mean Ocean Water, VSMOW), and wherein total level of all eight heavy isotopologues comprising at least one heavy isotopes ²H, ¹⁷O, or ¹⁸O is about 0.2683% (e.g. 0.199983% ¹H²H¹⁸O, 0.0372% ¹H₂¹⁷O, 0.031069% ¹H²H¹⁶O, 0.0000623% ¹H²H¹⁸O, and 0.0000116% ¹H²H¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, δ, relative to the international VSMOW standard. The Earth water maximally enriched by major light water isotopologue ¹H₂¹⁶O was founded in Antarctica (Standard Light Antarctic Precipitation, SLAP), wherein said δ-values of residual heavy isotopes are δ²H −415.5‰, δ¹⁷O −28.1‰, and δ¹⁸O −53.9‰ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50. Thus, water with the abundance of light water isotopologue ¹H₂¹⁶O more than 99.757 molecular % is not found in nature.

Deuterium depleted water (DDW) is known from the art and is prepared from natural water by industrial procedures providing depletion of heavy isotopologues comprising deuterium, predominantly of ¹H²H¹⁶O (HOD). Since total levels of deuterium-comprising isotopologues in water is below 0.031 molecular %, complete depletion of natural water of deuterium-comprising isotopologues provides water enriched by light water isotopologue ¹H₂¹⁶O to the level never more than 99.76 molecular %. Thus, water with level of light water isotopologue ¹H₂¹⁶O more than 99.76% is unknown from the art and can be prepared in industrial scale by methods providing depletion of natural water of heavy isotopologues comprising isotopes ¹⁷O and ¹⁸O.

We discovered that water with isotopologue ¹H₂¹⁶O level more than 99.76 molecular % is useful for decreasing postprandial blood glucose excursion in mammals in need thereof.

It is an object of the present invention to provide a method for decreasing postprandial glucose excursion in a mammal in need thereof comprising a step of administering to said mammal an effective amount of water comprising from 99.76 to 99.99 molecular % of isotopologue ¹H₂¹⁶O.

It is an object of the present invention to provide a medical food for decreasing postprandial glucose excursion in a mammal in need thereof which comprises water comprising from about 99.76 to about 99.99 molecular % of isotopologue ¹H₂¹⁶O.

It is an object of the present invention to provide the use of water comprising from about 99.76 to about 99.99 molecular % of isotopologue 1H₂¹⁶O for the manufacture of a medical food for decreasing postprandial glucose excursion in a mammal in need thereof.

### BRIEF DESCRIPTION OF THE DRAWING

FIG. 1 is a schematic side view of an apparatus for the manufacturing the water comprising from 99.76 to 99.99 molecular % of isotopologue ¹H₂¹⁶O.

### DISCLOSURE OF INVENTION

The present invention provides a method for decreasing postprandial glucose excursion in a mammal in need thereof comprising a step of administering to said mammal an effective amount of water comprising from 99.76 to 99.99 molecular % of isotopologue ¹H₂¹⁶O at 0 to 240 min from the start of ingestion of a meal.

As used herein, the term “postprandial glucose excursion” refers to the difference between postprandial and pre-prandial blood glucose levels.

As used herein, the term “postprandial glucose” refers to blood glucose level after eating. Postprandial glucose can be measured at 0 to 240 min from the start of ingestion of a meal. Preferably, postprandial glucose is measured two hours after the start of a meal.

As used herein, the term “pre-prandial glucose” refers to blood glucose level just before eating beginning.

As used herein, the term “decreasing” means decreasing a magnitude of glucose excursion, a peak value of glucose excursion, or an area under curve of glucose excursion in a time.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H²H¹⁶O, ¹H₂¹⁸O.

The water of the invention comprising from 99.76 to 99.99 molecular % of isotopologue ¹H₂¹⁶O can be prepared by a variety of industrial procedures. Such procedures include, but are not limited to, burning molecular hydrogen with molecular oxygen with desired low heavy isotope content, or industrial procedures providing purification of natural water of heavy isotopologues comprising heavy isotopes ²H, ¹⁷O, and ¹⁸O. Preferably, the water of the invention is prepared by highly-effective distillation of natural water.

According to present invention, the water of the invention comprises from 99.76 to 99.99 molecular % of isotopologue ¹H₂¹⁶O and up to 100 molecular % of residual isotopologues. As used herein, the term “residual isotopologues” refers to ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O. In the invention, relative amounts of particular heavy isotopologues could vary depending upon the procedure of the preparing the water of the invention, but the total sum of residual isotopologues formed by heavy isotopes ²H, ¹⁷O, ¹⁸O should not exceed 0.01 to 0.24 molecular %. The amounts of heavy isotopes in the residual isotopologues could vary from 0.01 ppm to 155 ppm for ²H, 1 to 360 ppm for ¹⁷O, and 1 to 2000 ppm for ¹⁸O, but the total sum of the residual isotopologues formed by these amounts of heavy isotopes should not exceed 0.01 to 0.24%.

In practicing the method of the invention, the effective amount of water comprising from 99.76 to 99.99 molecular % of isotopologue ¹H₂¹⁶O can be administered in a variety of routes including oral (e.g. through gastrointestinal tract or oral mucosa), intranasal, topical, rectal, by inhalation spray, or parenteral (e.g. subcutaneous, intravenous, or intramuscular injections). Preferably, the effective amount water comprising from 99.76 to 99.99 molecular % of isotopologue ¹H₂¹⁶O is administered orally.

Preferably, the effective amount of the water comprising from about 99.76 to about 99.99 molecular % of isotopologue ¹H₂¹⁶O is 0.1 to 20 g/kg body weight of a mammal.

As used herein, the term “meal” refers to a portion of food taken at a particular time for the satisfaction of appetite.

As used herein, the term “mammal” refers to any mammal. Nonexclusive examples of such mammals include, but are not limited to, animals such as a dog, a cat, and a horse and a human. Preferably, the mammal is a human.

In practicing the method of the invention, the effective amount of water comprising from 99.76 to 99.99 molecular % of isotopologue ¹H₂¹⁶O isotopologues can be administered in a variety of different dosage forms, i.e., they may be formulated in the form of solutions, spray, liquid aerosols, elixirs, syrups, and the like. Ingredients that can be used for preparing dosage forms of the invention may include, but are not limited to, buffering agents (such as phosphate buffer, carbonate buffer, tris buffer, tartrate buffer, borate buffer, acetate buffer, succinate buffer, or maleate buffer), colorants, flavorants, preservatives, antioxidants, surfactants, and etc.

In practicing the method of the invention, the effective amount of water comprising from 99.76 to 99.99 molecular % of isotopologue ¹H₂¹⁶O can be administered stepwise or simultaneously with other postprandial glucose regulators. Such postprandial glucose regulators include, but are not limited to, insulin, incretines (e.g. GLP-1 or GIP), exendines (e.g. exendine-4), sulphonylureas, repaglinide, and acarbose.

Further, the present invention provides a medical food for decreasing postprandial glucose excursion in a mammal in need thereof which comprises water comprising from about 99.76 to about 99.99 molecular % of isotopologue ¹H₂¹⁶O.

Further, the present invention provides the use of water comprising from about 99.76 to about 99.99 molecular % of isotopologue ¹H₂¹⁶O for the manufacture of a medical food for decreasing postprandial glucose excursion in a mammal in need thereof.

Preferably, the medical food for decreasing postprandial glucose excursion is drinking water or beverage. Preferably, the medical food of the invention is drinking water manufactured by saturation of the water of the invention with carbon dioxide or/and inorganic salts typically abandoned in natural drinking water. The examples of such salts include, but are not limited to, sodium chloride, sodium bicarbonate, calcium chloride, magnesium sulfate, etc.

Because of the method of the present invention, it is now possible to reduce postprandial glucose spikes and diminish postprandial hyperglycemia arising from overloading high-carbohydrate food during a meal in apparently healthy mammals. Also, it is now possible to reduce postprandial glucose spikes and diminish postprandial hyperglycemia arising from disturbances in glucose metabolism in mammals with metabolic syndrome, insulin resistance, impaired glucose tolerance or diabetes mellitus.

Because of decreasing postprandial glucose excursion, the invention is particularly useful for preventing risk of cardiovascular diseases and type 2 diabetes in apparently healthy mammals, mammals with impaired glucose tolerance and insulin resistance, and overweight or obese mammals. Also, the invention is particularly useful for preventing risk of cardiovascular diseases in diabetic mammals.

The following examples are presented to demonstrate the invention. The examples are illustrative only and are not intended to limit the scope of the invention in any way.

### EXAMPLE 1

This example demonstrates the method for producing the water of the invention.

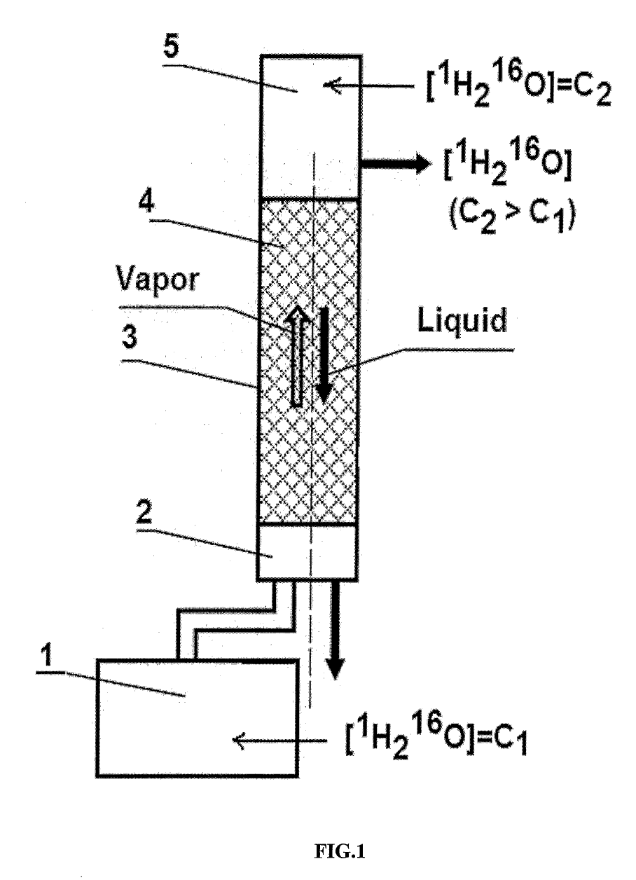

Water comprising from 99.76 to 99.99 molecular % of isotopologue ¹H₂¹⁶O is prepared by distillation of natural water comprising 99.73% of isotopologue ¹H₂¹⁶O with using the apparatus of FIG. 1 under temperature 60° C. and pressure 0.2 bars. The process of the distillation comprises evaporating natural water comprising 99.71% (C₁) of isotopologue ¹H₂¹⁶O in boiling means 1 to produce water vapor; supplying the water vapor to the bottom 2 of distillation column 3; carrying out vapor-liquid contact between a descending liquid and an ascending vapor mainly on the surface of the contact device 4 (e.g. structured or random packing) within the distillation column, at which time the liquid and the vapor flow in mutually opposite directions over the surface of the contact device along a main flow direction which is along a direction of the column axis; condensing water vapor with concentration of isotopologue ¹H₂¹⁶O from 99.76% to about 99.99% (C₂) on condenser 5 installed on upper bound of the distillation column 3; and collecting a part of condensate as condensed water comprising from 99.76% to 99.99% of isotopologue ¹H₂¹⁶O (C₂>C₁). Water comprising from 99.76 to 99.99 molecular % of isotopologue ¹H₂¹⁶O is used in the examples of the invention.

### EXAMPLE 2

This example demonstrates the method for decreasing postprandial glucose excursion.

Male Wistar rats received fructose-rich diet (66% fructose of total calorie intake) for 30 days before the experiment to induce insulin resistance. The rats were assigned into two groups: a control rats (n=10) and experimental rats (n=10). Glucose 1 g/kg was administered into stomach of rats with feeding tube to induce postprandial glycemic response. At 60 min since glucose loading, experimental rats received orally 5 ml water (99.85 molecular % of isotopologue ¹H₂¹⁶O). Control rats received 5 ml control water with natural isotopes content (99.73 molecular % of isotopologue ¹H₂¹⁶O). Postprandial blood glucose levels at 2 hours since the glucose loading were measured. Data are presented in Table 1 as postprandial glucose excursion mean ±SD (n=10) at 2 hours since the glucose loading. Table 1 demonstrates that water of the invention is effective for decreasing postprandial glucose excursion as compared to control.

### EXAMPLE 3

This example demonstrates the method for decreasing postprandial glucose excursion.

Overweight male human subject with mean BMI 29.6 kg/m² received standard test breakfast consisted of 75 g of hard-boiled eggs, 60 g of white bread, 10 g of butter, and 200 ml of fresh orange juice (400 kcal: 44% carbohydrate, 16% protein, and 40% fat). At 30 min since the start of the breakfast, the subject received additively 250 ml of water of the invention (99.80 molecular % of isotopologue ¹H₂¹⁶O) as the drink at days 1, 3, and 5, or 250 ml of control water (99.73 molecular % of isotopologue ¹H₂¹⁶O) as the drink at days 2, 4, and 6. Pre-prandial blood glucose levels were measured just before the breakfast and postprandial glucose levels were measured at 2 hours since the breakfast started. Data are presented in Table 2 as postprandial glucose excursion mean ±SD (n=3) at 2 hours since the breakfast started. Table 2 demonstrates that water of the invention is effective for decreasing postprandial glucose excursion as compared to control.

### EXAMPLE 4

Table 3 demonstrates the medical food (drinking water) for decreasing postprandial glucose excursion.

The medical food as described in Table 3 was manufactured as follows: salts (calcium chloride, magnesium chloride, and sodium bicarbonate) in amounts shown in Table 3 were dissolved in the water (99.99 molecular % of isotopologue ¹H₂¹⁶O) under room temperature and, then, resulted product was bottled in bottles of 330 ml volume.

TABLE 1
Postprandial Glucose Excursion.

| | | | Postprandial |
|---|---|---|---|
| | Pre-prandial | Postprandial | glucose excursion, |
| Group | glucose, mmol/L | glucose, mmol/L | mmol/L |
| Control | 6.9 ± 1.1 | 11.5 ± 1.7 | 4.6 |
| Experimental | 7.0 ± 0.9 | 7.4 ± 0.7* | 0.4 |

*Denotes statistically significant difference of control (p < 0.05)

TABLE 2
Postprandial Glucose Excursion

| | | | Postprandial |
|---|---|---|---|
| | Pre-prandial | Postprandial | glucose excursion, |
| | glucose, mmol/L | glucose, mmol/L | mmol/L |
| Control | 6.5 ± 0.4 | 8.9 ± 1.1 | 2.4 |
| water | | | |
| Water of the | 6.6 ± 0.3 | 7.4 ± 0.9* | 0.8 |
| invention | | | |

*Denotes statistically significant difference of control (p < 0.05)

TABLE 3
Medical food for decreasing postprandial glucose excursion.

| | Content, |
|---|---|
| | weight % |
| Water (99.99 molecular % of isotopologue ¹H₂¹⁶O) | 99.953 |
| Calcium chloride | 0.015 |
| Magnesium chloride | 0.007 |
| Sodium bicarbonate | 0.025 |

We claim:

1. A method for decreasing postprandial glucose excursion in a mammal in need thereof comprising a step of administering to said mammal an effective amount of water comprising from 99.76 to 99.99 molecular % of isotopologue ¹H₂¹⁶O at 0 to 240 min from the start of ingestion of a meal.

2. The method according to claim 1, wherein the effective amount water comprising from 99.76 to 99.99 molecular % of isotopologue ¹H₂¹⁶O is administered orally.

3. The method according to claim 1, wherein the effective amount of the water comprising from about 99.76 to about 99.99 molecular % of isotopologue ¹H₂¹⁶O is 0.1 to 20 g/kg body weight of a mammal.

4. The method according to claim 1, wherein the mammal is a human.

5. A medical food for decreasing postprandial glucose excursion in a mammal in need thereof which comprises water comprising from about 99.76 to about 99.99 molecular % of isotopologue ¹H₂¹⁶O.

6. The medical food according to claim 5, wherein the mammal is a human.

7. The use of water comprising from about 99.76 to about 99.99 molecular % of isotopologue ¹H₂¹⁶O for the manufacture of a medical food for decreasing postprandial glucose excursion in a mammal in need thereof.

8. The use according to claim 7, wherein the mammal is a human.
